(19) United States
(12) Patent Application Publication
(10) Pub. No.: US 2020/0046539 A1
(43) Pub. Date: Feb. 13, 2020

(54) BACK SUPPORT APPARATUS

(71) Applicant: Khadijeh Jawed, Corbin, KY (US)

(72) Inventor: Khadijeh Jawed, Corbin, KY (US)

(21) Appl. No.: 16/102,139

(22) Filed: Aug. 13, 2018

(51) Int. Cl. A61F 5/02

(52) U.S. Cl. CPC A61F 5/026; A61F 5/028

(57) ABSTRACT

A back support apparatus that includes: a first shoulder strap; a second shoulder strap; a horizontal connecting strap, where the horizontal connecting strap connects the first shoulder strap to the second shoulder strap; an means to adjust tension on the horizontal connecting strap; a first front shoulder pad; and a second front shoulder pad. The means to adjust includes an adjusting clip and tie strings, where the adjusting clip is on the horizontal connecting strap and tie strings are pulled to increase tension on the first shoulder strap and the second shoulder strap.

### BACKGROUND OF THE INVENTION

### Field of Invention

The present invention relates to a back support apparatus used to support the alignment of the upper torso of the individual.

### Description of Related Art

Many individuals suffer from back pain due to poor posture. This is normally caused by long periods of improper posture and due to various types of work conditions. Particularly, office workers hunch over a desk and/or a computer and therefore cause misalignment of the vertebra of the back. Continued poor posture can caused long term injury and/or pain. Some devices have been developed to address this problem. Many are difficult to use and lack the effectiveness to address this problem. It is the object of the present invention to provide a back support apparatus that helps to correct the posture of a user.

### SUMMARY OF THE INVENTION

The present invention relates to a back support apparatus that includes: a first shoulder strap; a second shoulder strap; a horizontal connecting strap, where the horizontal connecting strap connects the first shoulder strap to the second shoulder strap; an means to adjust tension on the horizontal connecting strap; a first front shoulder pad; and a second front shoulder pad. The means to adjust includes an adjusting clip and tie strings, where the adjusting clip is on the horizontal connecting strap and tie strings are pulled to increase tension on the first shoulder strap and the second shoulder strap.

### BRIEF DESCRIPTION OF DRAWINGS

FIG. 1 depicts a back view of a back support apparatus according to the present invention.

FIG. 2 depicts a front view of the shoulder supports of the back support apparatus according to the present invention.

### DETAILED DESCRIPTION

The present invention includes a back support apparatus that addresses hunched shoulders and helps to correct the upper back of the user. This apparatus is particularly helpful for correcting the upper vertebra of a user. The back support apparatus includes a horizontal connecting strap that extends between the shoulder blades of the user. This horizontal connecting strap attaches to a first shoulder strap on the first side and a second shoulder strap on a second side. An adjusting clip is provided on the horizontal connecting strap that includes tie strings that extend therefrom. The adjusting clip allows the user to adjust the shoulder straps accordingly. The shoulder straps extend across the front torso of the user and connect to a shoulder pad on each respective shoulder of the user. By adjusting the straps and tension using the horizontal strap connector and adjusting clip enables the user to apply pressure to extend or move the shoulders rearward to allow for the straightening of the upper vertebra of the user.

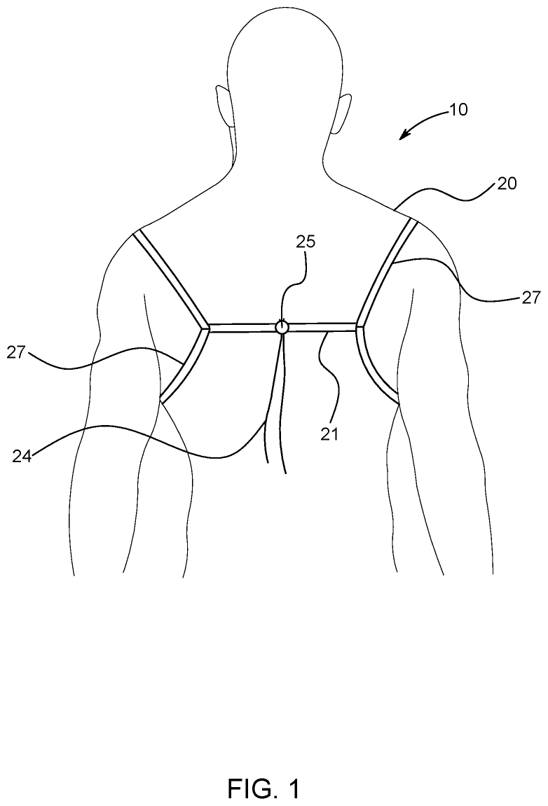

FIG. 1 depicts the back view of a back support apparatus 20 according to the present invention. This back support apparatus 20 includes a first shoulder strap and a second shoulder strap 27 that connect to a horizontal connecting strap 21. The horizontal connecting strap 21 extends between the shoulder blades of the user and an adjusting clip 25 is provided to adjust the tension between the respective shoulder straps. This adjustment in turn affects the posture of the user.

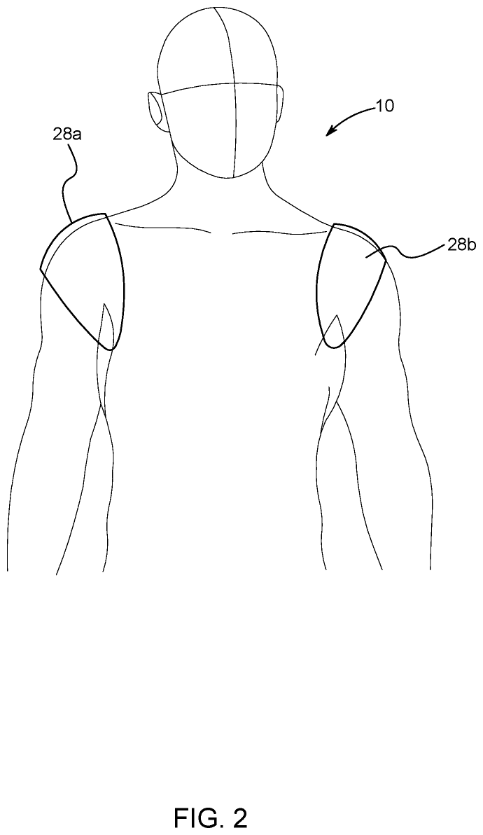

FIG. 2 shows the front view of the back support apparatus 20 where a first shoulder pad 28(a) and a second shoulder pad 28(b) are shown on each respective shoulder of the user. These pads connect to the shoulder straps 27 to provide a means to tension and move the shoulders backward and stretch the pectoral muscles of the user. The back support apparatus 20 corrects the posture of the user 10 as shown in FIGS. 1 and 2. The adjusted clip 25 allows the user to adjust the tension by pulling the tie strings 24 extending from the adjusting clip.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

What is claimed is:

1. A back support apparatus comprising
a. a first shoulder strap;
b. a second shoulder strap;
c. a horizontal connecting strap, where the horizontal connecting strap connects the first shoulder strap to the second shoulder strap;
d. an means to adjust tension on the horizontal connecting strap;
e. a first front shoulder pad; and
f. a second front shoulder pad.

2. The back support apparatus according to claim 1, where the means to adjust includes an adjusting clip and tie strings.

3. The back support apparatus according to claim 2, where the adjusting clip is on the horizontal connecting strap and tie strings are pulled to increase tension on the first shoulder strap and the second shoulder strap.
